(19) United States
(12) Patent Application Publication
(10) Pub. No.: US 2018/0338702 A1
(43) Pub. Date: Nov. 29, 2018

(54) SHIELDED RING RESONATOR STRUCTURE

(71) Applicant: EMPIRE TECHNOLOGY DEVELOPMENT LLC, Wilmington, DE (US)

(72) Inventors: Anthony J. Tagliaferro, Oakland, CA (US); Michael J. Schirle, San Jose, CA (US); Udai Singh, San Francisco, CA (US); John L. Unkovic, Santa Clara, CA (US); Gerard H. Vurens, Palo Alto, CA (US)

(73) Assignee: EMPIRE TECHNOLOGY DEVELOPMENT LLC, Wilmington, DE (US)

(21) Appl. No.: 15/606,328

(22) Filed: May 26, 2017

(51) Int. Cl. A61B 5/053; A61B 5/00

(52) U.S. Cl. CPC A61B 5/0537; A61B 5/4875

(57) ABSTRACT

Technologies are generally described to detect hydration using a shielded ring resonator structure. In some examples, a shielded ring resonator structure configured to be attached to a subject for hydration detection may include a ring resonator, one or more feed structures that couple signals into and out of the ring resonator, and a conductive shield structure that covers gaps between the ring resonator and the feed structure(s). The shield structure may be disposed between the gaps and the subject to reduce capacitive coupling between the gaps and the subject, thereby reducing current flow between the subject and the ring resonator and/or feed structure(s).

### BACKGROUND

Unless otherwise indicated herein, the materials described in this section are not prior art to the claims in this application and are not admitted to be prior art by inclusion in this section.

Ring resonators may be used to monitor moisture. In such cases, a ring resonator may be placed near or next to a water-containing sample, which affects the dielectric characteristics of the ring resonator. The moisture content of the sample may then be determined by evaluation of the dielectric characteristics of the ring resonator.

### SUMMARY

The present disclosure generally describes techniques to measure hydration using shielded ring resonator structures.

According to some examples, a method is provided to detect hydration in a subject The method may include coupling a shielded ring resonator structure to an exterior surface of the subject, where the shielded ring resonator structure includes a ring resonator and a feed structure separated from the ring resonator by a first gap and a second gap and configured to couple signals into and out of the ring resonator. The shielded ring resonator structure may further include a metallic shield structure conjured to cover the first and second gaps and a portion of the resonator. The method may further include applying a test signal to the feed structure to cause the shielded ring resonator structure to resonate with a first frequency characteristic and using the metallic shield structure to reduce a capacitive coupling, while the test signal is applied, between the exterior surface of the subject and the first and second gaps. The method may further include determining a quality factor of the shielded ring resonator structure that resonates and determining a hydration level of the subject based on the determined quality factor.

According to other examples, a shielded ring resonator structure configured to be coupled to an exterior surface of a subject to detect hydration of the subject is provided. The shielded ring resonator structure may include a ring resonator, a first feed structure separated from the ring resonator by a first gap and configured to coupe signals into the ring resonator, and a second feed s true tire separated from the ring resonator by a second gap and configured to couple signals out of the ring resonator. The shielded ring resonator structure may further include a first conductive shield structure configured to cover a first portion of the ring resonator and at least one of the first and second gaps such that a capacitive coupling between the exterior surface of the subject and at least one of the first and second gaps is reduced.

According to further examples, a system is provided to detect hydration in a subject. The system may include a shielded ring resonator structure and a test controller, where the test controller is coupled to the shielded ring resonator structure and the shielded ring resonator structure is configured to be coupled to an exterior surface of the subject. The shielded ring resonator structure may include a ring resonator, a first feed structure separated from the ring resonator by a first gap and configured to couple a first signal into the ring resonator, and a second feed structure separated from the ring resonator by a second gap and configured to couple a second signal out of the ring resonator. The shielded ring resonator structure may further include a first conductive shield structure configured to cover a first portion of the ring resonator and the first gap such that a capacitive cooping between the exterior surface of the subject and the first gap is reduced. The shielded ring resonator structure may further include a second conductive shield structure configured to cover a second portion of the ring resonator and the second gap such that a capacitive coupling between the exterior surface of the subject and the second gap is reduced. The test controller may be configured to apply the first signal to the shielded ring resonator structure to cause the ring resonator to resonate with a first frequency characteristic, determine a quality factor of the resonating ring resonator based on the second signal, and determine a hydration level of the subject based on the determined quality factor.

According to yet further examples, a method to manufacture a shielded ring resonator structure is provided. The method may include constructing a ring resonator on a substrate, disposing a first feed structure on the substrate such that a first gap separates the first feed structure and the ring resonator, where the first feed structure is configured to couple a first signal into the ring resonator, and disposing a second feed structure on the substrate such that a second gap separates the second feed structure and the ring resonator, where the second feed structure is configured to couple a second signal out of the ring resonator. The method may further include placing a first conductive shield structure to cover a first portion of the ring resonator and at least one of the first and second gaps such that a capacitive coupling between an exterior surface of a subject to which the shielded ring resonator structure is attached and at least one of the first and second gaps is reduced.

The foregoing summary is illustrative only and is not intended to be in any way limiting. In addition to the illustrative aspects, embodiments, and features described above, further aspects, embodiments, and features will become apparent by reference to the drawings and the following detailed description

### BRIEF DESCRIPTION OF THE DRAWINGS

The foregoing and other features of this disclosure will become more folly apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates a ring resonator structure configured to detect hydration in a sample;

FIG. 2 illustrates how a conductive layer on the exterior of a sample may defeat hydration detection using a ring resonator structure;

FIG. 3 illustrates how a shielded ring resonator structure may be used to detect hydration of a sample with an exterior conductive layer;

FIG. 4 illustrates a general purpose computing device, which may be used along with a shielded ring resonator structure to deled hydration in a subject;

FIG. 5 is a flow diagram illustrating an example method to detect hydration in a subject that may be performed by a computing device such as the computing device in FIG. 4; and

FIG. 6 illustrates a block diagram of an example computer program product, some of which are arranged in accordance with at least some embodiments described herein.

### DETAILED DESCRIPTION

In the following detailed desorption, reference is made to the accompanying drawings, which form a part hereof In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. The aspects of the present disclosure, as generally described herein, and illustrated in the figures, can be arranged, substituted, combined, separated, and designed in a wide variety of different configurations, all of which are explicitly contemplated herein.

This disclosure is generally drawn, inter alia, to methods, apparatus, systems, devices, and/or computer program products related to detection of hydration in a subject using a shielded ring resonator structure.

Briefly stated, technologies are generally described to detect hydration using a shielded ring resonator structure. In some examples, a shielded ring resonator structure configured to be attached to a subject for hydration detection may include a ring resonator, one or more feed structures that couple signals into and out of the ring resonator, and a conductive shield structure that covers gaps between the ring resonator anti the feed structures). The shield structure may be disposed between the gaps and the subject to reduce capacitive coupling between the gaps and the subject, thereby reducing current flow between the subject and the ring resonator and/or feed structure(s).

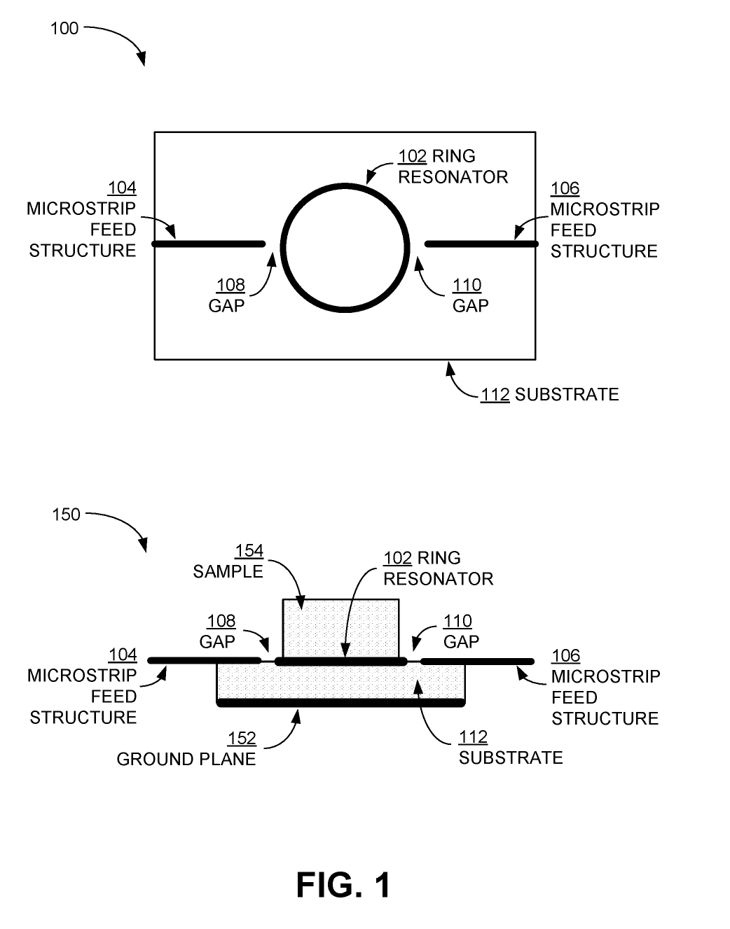

FIG. 1 illustrates a ring resonator structure configured to detect hydration in a sample.

Diagram 100 depicts a top-down view of an example ring resonator 102, and diagram 150 depicts aside view of the example ring resonator 102. The ring resonator 102 may be disposed on a substrate 112, which may be formed of a dielectric or isolative layer. A first microstrip feed structure 104 and a second microstrip feed structure 106 may also be disposed on the substrate 112 and positioned near the ring resonator 102. The first feed structure 104 may be separated from the ring resonator 102 by a first gap 108, and the second feed structure 106 may be separated from the ring resonator 102 by a second gap 110. In some embodiments, a ground plane 152 may be disposed on a surface of the substrate 112 opposite the ring resonator 102.

In some embodiments, the ring resonator 102 may be configured to resonate when signals of certain frequencies are coupled into the ring resonator 102. These frequencies, also known as resonance frequencies, may vary based on the circumference or diameter of the ring resonator 102. When a signal of a particular frequency is capacitively coupled into the ring resonator 102, for example via the first feed structure 104, the signal may propagate around the ring resonator 102, and may be measured or probed, for example using the second feed structure 106. A frequency characteristic of the ring resonator 102 (for example, a measured output of the ring resonator 102 as a function of input signal frequency) may then be determined by exciting the ring resonator 102 with signals of various frequencies and probing the response of the ring resonator 102. The frequency characteristic may be based cm electrical characteristics of the ring resonator 102 and its surrounding environment (for example, the substrate 112). Such electrical characteristics may include dielectric loss, conductor loss, and/or radiation loss.

When the environment of the ring resonator 102 changes, its frequency characteristic may also change. Accordingly, in some embodiments, the ring resonator 102 may be used to detect hydration in a sample 154 near or adjacent to the ring resonator 102. The electrical characteristics of the sample 154 may affect the environment of the ring resonator 102, and may cause the frequency characteristic of the ring resonator 102 to change. For example, if the sample 154 contains water, the ring resonator 102 may experience increased dielectric loss, leading to changes in the frequency characteristic. Accordingly, the frequency characteristic of the ring resonator 102 may vary based on the water content of the sample 154, and the frequency characteristic of the ring resonator 102 may therefore be used to determine the water content of the sample 154.

In some embodiments, the frequency characteristic of the ring resonator 102 may be used to determine a quality factor associated with the ring resonator 102. The quality factor (also known as Q factor) of the ring resonator 102 may be a dimensionless parameter that characterizes the bandwidth of the frequency response of the ring resonator 102 relative to a center frequency of the frequency response. The quality factor may be representative of the energy loss of a resonator such as the ring resonator 102 with respect to the resonator stored energy. A relatively larger quality factor corresponds to a relatively tower rate of energy loss, and vice-versa. For example, if the sample 154 has a relatively high water content, dielectric losses associated with the ring resonator 102 may be relatively high, resulting in a relatively low quality factor. In contrast, if the sample 154 has a relatively low water content, dielectric losses associated with the ring resonator 102 may be relatively low, resulting in a relatively high quality factor. Accordingly, in some embodiments a quality factor determined for the ring resonator 102 may be used to determine the water content of the sample 154.

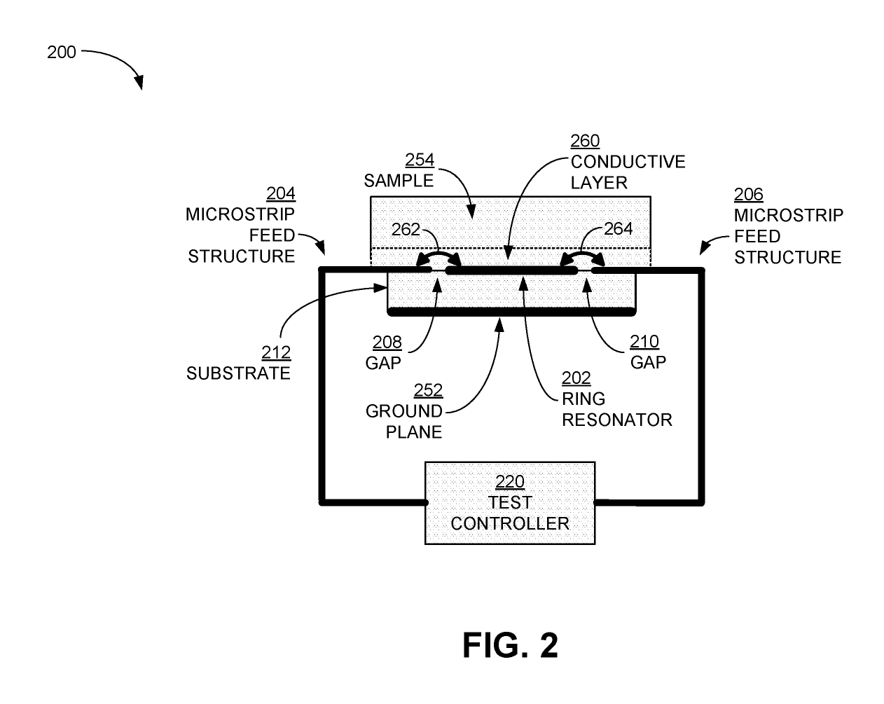

FIG. 2 illustrates how a conductive layer on the exterior of a sample may defeat hydration detection using a ring resonator structure.

Diagram 200 depicts aside view of a ring resonator structure, similar to diagram 150. The ring resonator structure may include a ring resonator 202, a first microstrip feed structure 204, and a second microstrip feed structure 206, all of which may be mounted on a substrate 212 having a ground plane 252. The ring resonator 202 may be separated from the first feed structure 204 by a first gap 208 and may be separated from the second feed structure 206 by a second gap 210. The first and second feed structures 204 and 206 may be coupled to a test controller 220 configured to couple test signals into and out of the ring resonator 202, for example to determine the frequency characteristic and/or quality factor of the ring resonator 202.

The ring resonator 202 may be used to measure the hydration of a sample 254 as described above in FIG. 1. However, in some situations the surface of the sample 254 may include a conductive layer 260. For example, the sample 254 may be a test subject such as a human being or an animal, the ring resonator structure may be attached or affixed to the skin of the test subject, and the conductive layer 260 may be sweat (or other electrically conductive substance) on the skin of the test subject. In these situations, the test signals from the test controller 220 may couple directly to the conductive layer 260, thereby at least partially bypassing the gaps 208 and 210. For example, an electrical current 262 may flow between the first feed structure 204 and the ring resonator 202 via the conductive layer 260, and an electrical current 264 may flow between the ring resonator 202 and the second feed structure 206 via the conductive layer 260. In some cases, the test signals may even bypass the ring resonator 202. Even if an isolative layer is disposed between the sample and the ring resonator structure, the conductive layer 260 may still capacitively couple with the feed structures 204 and 206, which may prevent or otherwise degrade accurate measurement of the hydration of the sample 254.

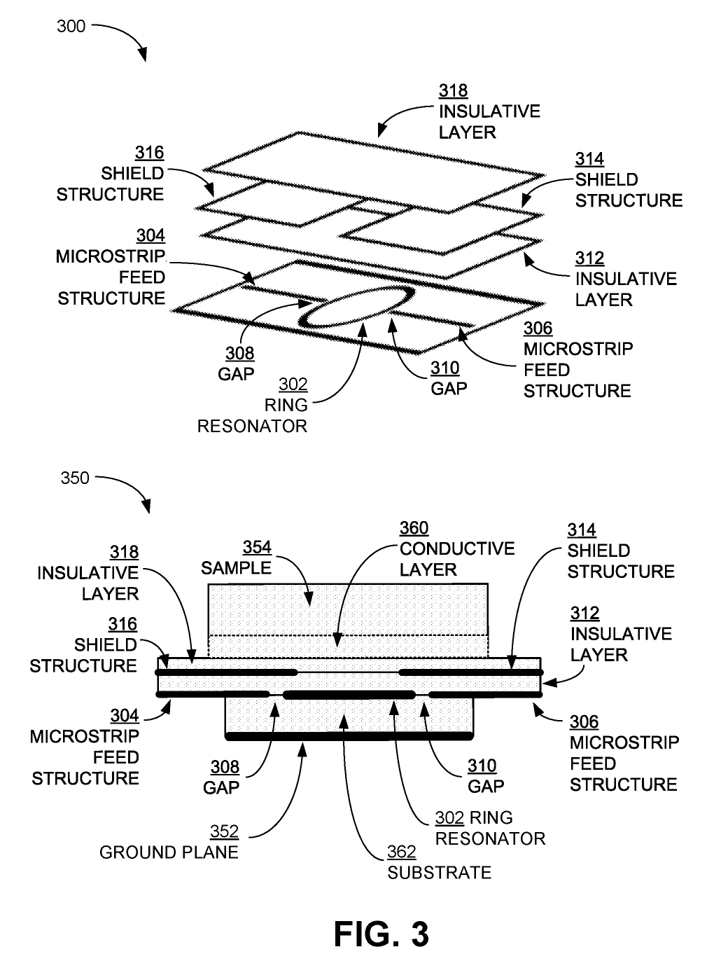

FIG. 3 illustrates how a shielded ring resonator structure may be used to detect hydration of a sample with an exterior conductive layer, arranged to accordance with at least some embodiments described herein.

Diagram 300 depicts an isometric and exploded view of a shielded ring resonator structure, and diagram 350 depicts a side view of the shielded rim; resonator structure adjacent and/or attached to a sample 354 having a conductive layer 360, which may be similar to the sample 254 and the conductive layer 260 in FIG. 2. The shielded ring resonator structure may include a ring resonator 302, a first microstrip feed structure 304, and a second microstrip feed structure 306, all disposed on a substrate 362. Similar to the structures depicted to FIGS. 1 and 2, the ring resonator 302 may be separated from the first feed structure 304 by a first gap 308 and may be separated from the second feed structure 306 by a second gap 310. The ring resonator 302 may be constructed on the substrate 362 or placed on the substrate 362 after construction, and to some embodiments, may have a diameter ranging from about 3 millimeters to about 50 millimeters, depending on the desired resonant frequencies.

The shielded ring resonator structure may further include a first shield structure 314 and a second shield structure 316. The first and second shield structures 314/316 may be electrically conductive, and to some embodiments may include or be formed of a metallic material, such as aluminum, copper, or any other suitable metal The first and second shield structures 314/316 may be sized, shaped, and disposed to at feast partially cover the first and second gaps 308/310, thereby preventing or otherwise reducing capacitive coupling between the first and second gaps 308/310 and the sample 354. For example, the first shield structure 314 may be sized, shaped, and disposed to cover the first gap 308 and at feast a portion of the ring resonator 302, and the second shield structure 316 may be sired, shaped, and disposed to cover the second gap 310 and at feast a portion of the ring resonator 302. In some embodiments, the first shield structure 314 and/or the second shield structure 316 may have a width between 3 mm and 100 mm, a length between 3 mm and 100 mm, a generally rectangular shape, and a thickness between 1 μm and 2500 μm.

The first shield structure 314 and/or the second shield structure 316 may be electrically isolated from each other, electrically coupled to each other, electrically coupled to a reference potential, such as a ground plane 352, or floating (e.g., not coupled to a specific reference potential). In some embodiments, the first and second shield structures 314/316 may be part of a stogie shield structure. The first and second shield structures 314/316, while configured to cover the first and second gaps 308/310, may be configured to leave at feast a portion of the ring resonator 302 uncovered, for example to facilitate coupling of the ring resonator 302 to the sample 354.

Referring back to the ring resonator structure of FIG. 1, if the user puts his/her finger on either or both of the first and second gaps 108/110 between the first and second microstrip feed structures 104/106 and the ring resonator 102 (instead of placing the finger on the center of the ring resonator 102), then the capacitance between the finger and the microstrip feed structures 104/106 and the ring resonator 102 could provide another path for electrical current(s) to travel through. Such electrical current(s) could affect the performance of the ring resonator structure of FIG. 1. To prevent or otherwise reduce this electrical coupling/capacitance between the user's finger and microstrip feed structure(s) and a ring resonator, the embodiment of the ring resonator structure of FIG. 3 provides the first and second shield structures 314/316, which may be positioned so that they at least partially cover the first/second gaps 308/310 between the first and second microstrip feed structures 304/306 and the ring resonator 302. In some embodiments, the first and second shield structures 314/316 need not cover most of the ring resonator 302, such that at least some or most of the ring resonator 302 is exposed between the first and second shield structures 314/316. With the placement of the first and second shield structures 314/316 as shown and described herein, the first and second shield structures 314/316 can prevent (or otherwise reduce) the user from affecting the current(s) at the sensitive positions of the first/second gaps 308/310.

The shielded ring resonator structure may further include a first insulative layer 312 and a second insulative layer 318. The first insulative layer 312 may be disposed between (a) the shield structures 314/316 and (b) the feed structures 304/306 and the ring resonator 302, and may be configured to electrically insulate the conductive shield structures 314/316 from either or both the feed structures 304/306 and the ring resonator 302. The second insulative layer 318 may be disposed between the shield structures 314/316 and the sample 354, and may be configured to electrically insulate the conductive shield structures 314/316 from the sample 354, to prevent or otherwise reduce electrical current flow between the conductive shield structures 314/316 through the conductive layer 360. For example, if the user touched both shield structures 314/316 and if the second insulative layer 318 is absent, then the currents) could travel directly through the user's finger and not through the ring resonator 302 at all. To reduce or prevent this current path, the second insulative layer 318 is provided in one embodiment

The insulative layers 312/318 may be formed of any suitable electrically insulative material For example, in embodiments where the shielded ring resonator structure is attached to a human subject, the insulative layers 312/318 may include medical tape and/or low-allergen-content tape. In some embodiments, the insulative byers 312/318 may have a thickness of between 1 μμm and 2500 μm. The insulative byers 312/318 may have any suitable shape so as to conform to the footprint of the shielded mg resonator, to adequately provide insulation between the various components described above, etc.

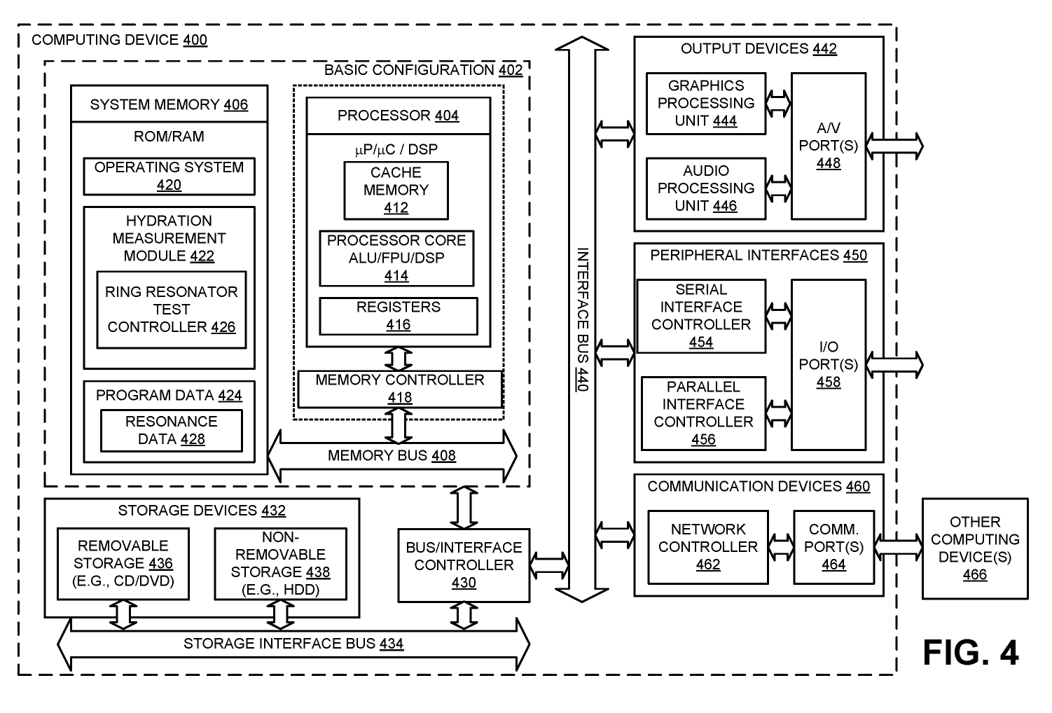

FIG. 4 illustrates a general purpose computing device, which may be used along with a shielded ring resonator structure to detect hydration in a subject, arranged to accordance with at least some embodiments described herein.

For example, a computing device 400 may be coupled to the shielded ring resonator of FIG. 3 and may be used to measure hydration in a subject as described herein. In an example basic configuration 402, the computing device 400 may include one or more processors 404 and a system memory 406. A memory bus 408 may be used to communicate between the processor 404 and the system memory 406. The basic configuration 402 is illustrated to FIG. 4 by those components within the toner dashed line.

Depending on the desired configuration, the processor 404 may be of any type, including but not limited to a microprocessor (μP), a microcontroller (μC), a digital signal processor (DSP), or any combination thereof The processor 404 may include one more levels of caching, such as a cache memory 412, a processor core 414, and registers 416. The example processor core 414 may include an arithmetic logic unit (ALU), a floating point unit (FPU), a digital signal processing core (DSP Core), or any combination thereof An example memory controller 418 may also be used with the processor 404, or to some implementations the memory controller 418 may be an internal part of the processor 404.

Depending on the desired configuration, the system memory 406 may be of any type including but not limited to volatile memory (such as RAM), non-volatile memory (such as ROM, flash memory, etc.) or any combination thereof the system memory 406 may include an operating system 420, a hydration measurement module 422, and program data 424. The hydration measurement module 422 may include a ring resonator test controller 426 to provide and receive signals from a shielded ring resonator stricture as described herein. The program data 424 may include, among other data, resonance data 428, other hydration-related data, or the like.

The computing device 400 may haw additional features or functionality, and additional interfaces to facilitate communications between the basic configuration 402 and any desired devices and interfaces. For example. a bus/interface controller 430 may be used to facilitate communications between the basic configuration 402 and one or more data storage devices 432 via a storage interface bus 434. The data storage devices 432 may be one or more removable storage devices 436, one or more non-removable storage devices 438, or a combination thereof Examples of the removable storage and the non-removable storage devices include magnetic disk devices such as flexible disk drives and hard-disk drives (HDDs), optical disk drives such as compact disc (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer readable instructions, data structures, program modules, or other data.

The system memory 406, the removable storage devices 436 and the non-removable storage devices 438 are examples of computer storage media. Computer storage media includes, but is not linked to, RAM, ROM, EEPROM, flash memory or other memory technology, CD-ROM, digital versatile disks (DVDs), solid state drives, or other optical storage, magnetic cassettes, magnetic tape, magnetic disk storage or other magnetic storage devices, or arty other medium which may be used to store the desired information and which may be accessed by the computing device 400. Any such computer storage media may be part of the computing device 400.

The computing device 400 may also include an interface bus 440 for facilitating communication from various interface devices (e.g., one or more output devices 442, one or more peripheral interfaces 444, and one or more communication devices 466) to the basic configuration 402 via the bus/interface controller 430. Some of the example output devices 442 include a graphics processing unit 448 and an audio processing unit 450, which may be conjured to communicate to various external devices such as a display or speakers via one or more A/V ports 452. One or more example peripheral interfaces 444 may include a serial interface controller 454 or a parallel interface controller 456, which may be configured to communicate with external devices such as input devices (e.g., keyboard, mouse, pen, voice input device, touch input device, etc.) or other peripheral devices (e.g., printer, scanner, etc.) via one or more I/O ports 458. In one embodiment, at least one of the I/O ports 458 and/or other port(s) of the computing device 400 can be used to couple the computing device 400 with the shielded ring resonator of FIG. 3, for communication of data, control information, instructions, signals, etc. An example communication device 466 includes a network controller 460. which may be arranged to facilitate communications with one or more other computing devices 462 over a network communication link via one or more communication ports 464, The one or more other computing devices 462 may include servers at a datacenter, customer equipment, and comparable devices.

The network communication link may be one example of a communication media. Communication media may be embodied by computer readable instructions, data structures, program modules, or other data in a modulated data signal such as a carrier wave or other transport mechanism, and may include any information delivery media. A “modulated data signal” may be a signal that has one or more of its characteristics set or changed to such a manner as to encode information to the signal By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

The computing device 400 may be implemented as a part of a general purpose or specialized server, mainframe, or similar computer that includes any of the above functions. The computing device 400 may also be implemented as a personal computer including both laptop computer and non-laptop computer conjurations.

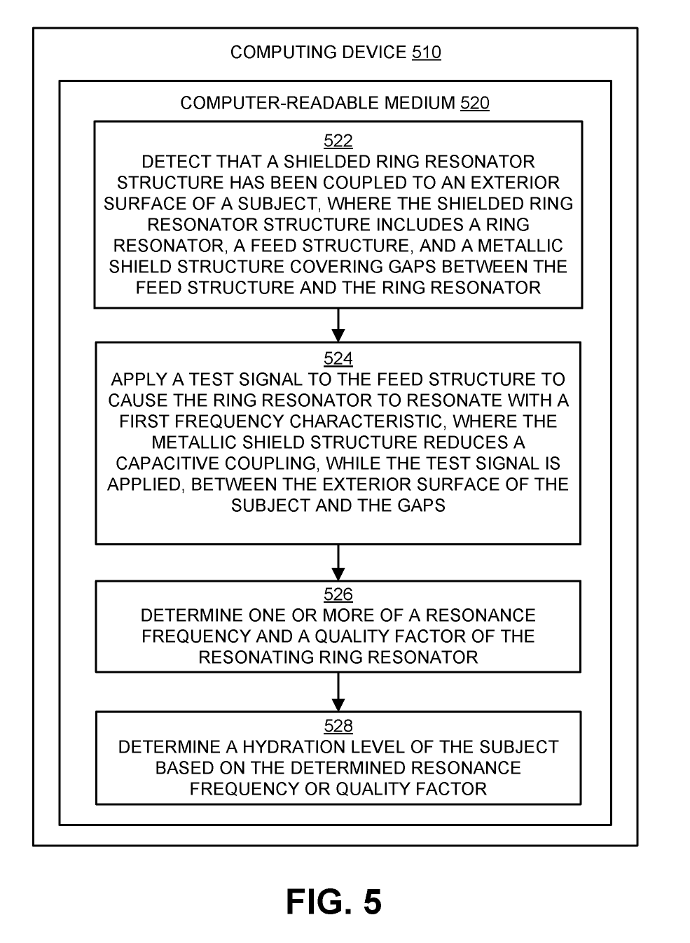

FIG. 5 is a flow diagram illustrating an example method to detect hydration to a subject that may be performed by a compiling device such as the computing device to FIG. 4, arranged in accordance with at least some embodiments described herein.

Example methods may include one or more operations, functions, or actions as illustrated by one or more of blocks 522, 524,526. and/or 528, and may to some embodiments be performed by a computing device such as the computing device 500 in FIG. 5. The operations described to the blocks 522-528 may also be stored as computer-executable instructions to a tangible and non-transitory computer-readable medium such as a computer-readable medium 520 of a computing device 510.

An example process to detect hydration in a subject may begin with block 522. “DETECT THAT A SHIELDED RING RESONATOR STRUCTURE HAS BEEN COUPLED TO AN EXTERIOR SURFACE OF A SUBJECT. WHERE THE SHIELDED RING RESONATOR STRUCTURE INCLUDES A RING RESONATOR, A FEED STRUCTURE, AND A METALLIC SHIELD STRUCTURE COVERING GAPS BETWEEN THE FEED STRUCTURE AND THE RING RESONATOR”, where a test controller may detect that a shielded ring resonator structure as described above has been coupled to an exterior surface of a subject, such as a human being, an animal, or some other sample, for hydration monitoring For example, the test controller may detect a change to output from the shield ring resonator structure indicative of coupling to a subject. The various components of the shielded ring resonator may be arranged/coupled together such as described above with respect to FIG. 3.

Block 522 may be followed by block 524, “APPLY A TEST SIGNAL TO THE FEED STRUCTURE TO CAUSE THE RING RESONATOR TO RESONATE WITH A FIRST FREQUENCY CHARACTERISTIC, WHERE THE METALLIC SHIELD STRUCTURE REDUCES A CAPACITIVE COUPLING, WHILE THE TEST SIGNAL IS APPLIED, BETWEEN THE EXTERIOR SURFACE OF THE SUBJECT AND THE GAPS”, where the test controller may apply a test signal to a feed structure of the shielded ring resonator structure. For example, the test controller may apply a test signal to a first feed structure, which may to turn may couple the test signal into the ring resonator. The test signal may be a signal of a single frequency, a combination of signals having multiple frequencies, a series of signals each having a different frequency, or one or more broadband signals, each spanning a frequency range. In some embodiments, the frequencies of the test signal may be resonant frequencies of the ring resonator, and the test signal may cause the ring resonator to resonate. When the test signal is applied, the metallic shield structure may act to reduce or prevent capacitive coupling between the exterior surface of the subject and the gaps between the feed structure and the ring resonator, as described above to FIG. 3.

Block 524 may be followed by block 526, “DETERMINE ONE OR MORE OF A RESONANCE FREQUENCY AND A QUALITY FACTOR OF THE RESONATING RING RESONATOR”, where the test controller may measure or otherwise determine a frequency characteristic of the resonating ring resonator (for example, via a microstrip feed structure or another microstrip feed structure) and/or determine a quality factor based on the measured frequency characteristic, as described above.

Block 526 may be followed by block 528. “DETERMINE A HYDRATION LEVEL OF THE SUBJECT BASED ON THE DETERMINED RESONANCE FREQUENCY OR QUALITY FACTOR”, where the test controller may use the resonance frequency or the quality factor determined at block 528 to determine a hydration level of the subject. For example, quality factor may be inversely related to hydration level, and the test controller may use an algorithm, calibrated values, a lookup table, or any suitable technique to derive a hydration level from the determined quality factor. In some embodiments, the test controller may determine a change to hydration instead of or in addition to a specific hydration level

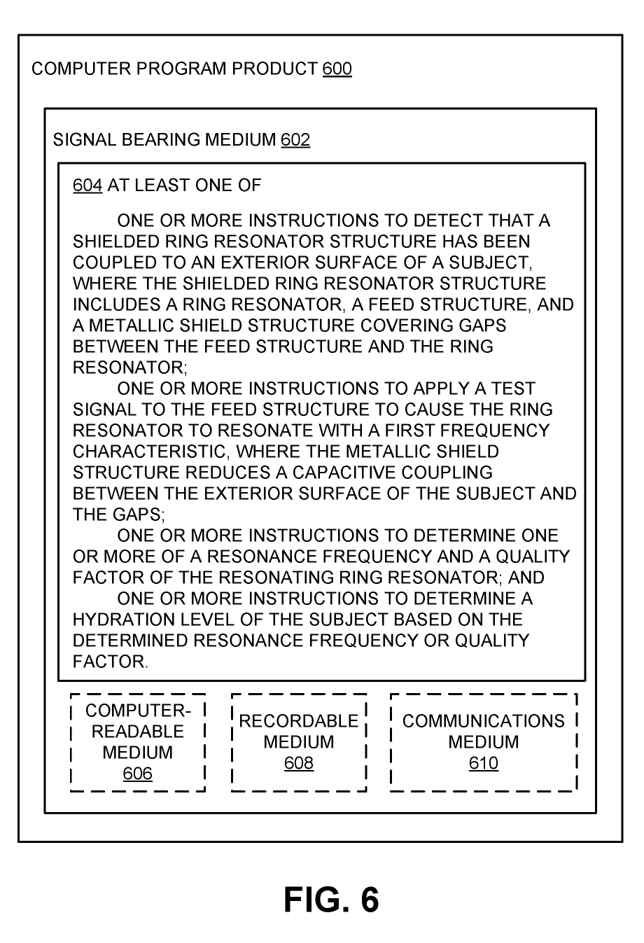

FIG. 6 illustrates a block diagram of an example computer program product, arranged to accordance with at least some embodiments described herein.

In some examples, as shown to FIG. 6, a computer program product 600 may include a signal bearing medium 602 that may also include one or more machine readable instructions 604 that, when executed by, for example, a processor may provide the functionality described herein. Thus, for example, referring to the processor 404 to FIG. 4, the hydration measurement module 422 may undertake one or more of the tasks shown to FIG. 6 to response to the instructions 604 conveyed to the processor 404 by the medium 602 to perform actions associated with detecting hydration to a subject as described herein. Some of those instructions may include, for example, instructions to detect that a shielded ring resonator structure has been copied to an exterior surface of a subject, where the shielded ring resonator structure includes a ring resonator, a feed structure, and a metallic shield structure covering gaps between the feed structure and the ring resonator. The instructions may also include instructions to apply a test signal to the feed structure to cause the ring resonator to resonate with a first frequency characteristic, where the metallic shield structure reduces a capacitive coupling between the exterior surface of the subject and the first and second gaps, determine a quality factor of the resonating ring resonator, and/or determine a hydration level of the subject based on the determined quality factor, according to some embodiments described herein.

In some implementations, the signal bearing media 602 depicted to FIG. 6 may encompass tangible and non-transitory computer-readable media 606, such as, but not limited to, a hard disk drive, a solid state drive, a compact disc (CD), a digital versatile disk (DVD), a digital tape, memory, etc. In some implementations, the signal bearing media 602 may encompass recordable media 607, such as, but not limited to memory, read/write (R/W) CDs, R/W DVDs. etc. In some implementations, the signal bearing media 602 may encompass communications media 610, such as, but not limited to, a digital and/or an analog communication medium (e.g., a fiber optic cable, a waveguide, a wired communications link, a wireless communication link, etc.). Thus, for example, the program product 600 may be conveyed to one or more modules of the processor 404 by an RF signal bearing medium, where the signal bearing media 602 is conveyed by the wireless communications media 610 (e.g., a wireless communications medium conforming with the IEEE 802.11 standard).

According to some examples, a method is provided to detect hydration in a subject. The method may include coupling a shielded ring resonator structure to an exterior surface of the subject, where the shielded ring resonator structure includes a ring resonator and a feed structure separated from the ring resonator by a first gap and a second gap and conjured to couple signals into and out of the ring resonator. The shielded ring resonator structure may further include a metallic shield structure conjured to cover the first and second gaps and a portion of the resonator. The method may further include applying a test signal to the feed structure to cause the shielded ring resonator structure to resonate with a first frequency characteristic and using the metallic shield structure to reduce a capacitive coupling, while the test signal is applied, between the exterior surface of the subject and the first and second gaps. The method may further include determining a quality factor of the shielded ring resonator structure dial resonates and determining a hydration level of the subject based on the determined quality factor.

According to some embodiments, coupling the shielded ring resonator structure to the exterior surface of the subject may include disposing an insulative layer between the metallic shield structure and the exterior surface of dx subject and/or affixing the shielded ring resonator structure to a skin of a human being. The insulative layer may include a medical tape and/or a low-allergen-content tape.

According to other examples, a shielded ring resonator structure conjured to be coupled to an exterior surface of a subject to detect hydration of the subject is provided. The shielded ring resonator structure may include a ring resonator, a first feed structure separated from the ring resonator by a first gap and conjured to couple signals into the ring resonator, and a second feed structure separated from the ring resonator by a second gap and configured to couple signals out of the ring resonator. The shielded ring resonator structure may timber include a first conductive shield structure configured to cover a first portion of the ring resonator and at least one of the first and second gaps such that a capacitive coupling between the exterior surface of the subject and at least one of the first and second gaps is reduced.

According to some embodiments, the first conductive shield structure may be configured to cover both of the first and second gaps. The shielded ring resonator structure may further include a second conductive shield structure, where the first conductive shield structure is configured to cover the first portion of the ring resonator and the first gap and where the second conductive shield structure is configured to cover a second portion of the ring resonator and the second gap. The first conductive shielding structure may be coupled to a reference potential, and may include aluminum or copper. The shielded ring resonator structure may further include an insulative layer disposed between the first conductive shield structure and the first gap and/or the second gap, and may further include another insulative layer disposed between the first conductive shield structure and the exterior surface of the subject The insulative layer(s) may include a medical tape and/or a low-allergen-content tape. The first conductive shield structure may have a generally rectangular shape and may have a width to a range between about 3 mm and about 100 mm, a length to a range between about 3 mm and about 100 mm, and a thickness to a range between about 1 μm and about 2500 μm.

According to further examples, a system is provided to detect hydration to a subject The system may include a shielded ring resonator structure and a test controller, where the test controller is coupled to the shielded ring resonator structure and the shielded ring resonator structure is configured to be copied to an exterior surface of the subject. The shielded ring resonator structure may include a ring resonator, a first feed structure separated from the ring resonator by a first gap and conjured to couple a first signal into the ring resonator, and a second feed structure separated from the ring resonator by a second gap and conjured to couple a second signal out of the ring resonator. The shielded ring resonator structure may further include a first conductive shield structure configured to cover a first portion of the ring resonator and the first gap such that a capacitive coupling between the exterior surface of the subject and the first gap is reduced. The shielded ring resonator structure may further include a second conductive shield structure conjured to cover a second portion of the ring resonator and the second gap such that a capacitive coupling between the exterior surface of the subject and the second gap is reduced. The test controller may be conjured to apply the first signal to the shielded ring resonator structure to cause the ring resonator to resonate with a first frequency characteristic, determine a quality factor of the resonating ring resonator based on the second signal, and determine a hydration level of the subject based on the determined quality factor.

According to some embodiments, the first conductive shield structure may be coupled to a reference potential coupled to the second conductive shield structure, and/or include aluminum or copper. The system may further include an insulative layer disposed between the first conductive shield structure and the first gap and/or the second gap, and may further include another insulative layer disposed between the first conductive shield structure and the exterior surface of the subject The insulative layer(s) may include a medical tape and/or a low-allergen-content tape. The subject may be a human being and the exterior surface may be skin.

According to yet further examples, a method to manufacture a shielded ring resonator structure is provided. The method may include constructing a ring resonator on a substrate, disposing a first feed structure on the substrate such that a first gap separates the first feed structure and the ring resonator, where the first feed structure is conjured to couple a first signal into the ring resonator, and disposing a second feed structure on the substrate such that a second gap separates the second feed structure and the ring resonator, where the second feed structure is conjured to couple a second signal out of the ring resonator. The method may further include placing a first conductive shield structure to cover a first portion of the ring resonator and at least one of the first and second gaps such that a capacitive coupling between an exterior surface of a subject to which the shielded ring resonator structure is attached and at least one of the first and second gaps is reduced.

According to some embodiments, the method may further include placing an insulative layer between the first conductive shield structure and the first gap and/or the second gap. The method may further include placing an insulative layer between the first conductive shield structure and the exterior surface of the subject

There are various vehicles by which processes and/or systems anchor other technologies described herein may be effected (e.g., hardware, software, and/or firmware), and that the preferred vehicle may vary with the context to which the processes and/or systems and/or other technologies are deployed. For example, if an implementer determines that speed and accuracy are paramount, the implementer may opt for a mainly hardware and/or firmware vehicle; if flexibility is paramount, the topic mentor may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, each function and/or operation within such block diagrams, flowcharts, or examples may be implemented, individually and/or collectively, by a wide range of hardware, software, firmware, or virtually any combination thereof In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or to part, may be equivalently implemented to integrated circuits, as one or more computer programs executing on one or more computers (e.g., as one or more programs executing on one or more computer systems), as one or more programs executing on one or more processors (e.g., as one or more programs executing on one or more microprocessors), as firmware, or as virtually any combination thereof and that designing the circuitry and/or writing the code for the software and/or firmware is possible to light of this disclosure.

The present disclosure is not to be limited to terms of the particular embodiments described in this application, which are intended as illustrations of various aspects. Many modifications and variations can be made without departing from its spirit and scope. Functionally equivalent methods and apparatuses within the scope of the disclosure, to addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled. The terminology used herein is for the purpose of describing particular embodiments only, and is not intended to be limiting.

In addition, the mechanisms of the subject matter described herein are capable of be tog distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a compact disc (CD), a digital versatile disk (DVD), a digital tape, a computer memory, a solid state drive, etc.; and a transmission type medium such as a digital and/or an analog communication medium (e.g. a fiber optic cable, a waveguide, a wined communications link, a wireless communication link, etc.).

Those skilled to the art will recognize that k is common within the art to describe devices and/or processes in the fashion set forth herein, and thereafter use engineering practices to integrate such described devices and/or processes into data processing systems. That is, at least a portion of the devices and/or processes described herein may be integrated into a data processing system via a reasonable amount of experimentation. A data processing system may include one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops.

A data processing system may be implemented utilizing any suitable commercially available components, such as those found in data computing/communication and/or network computing/communication systems. The herein described subject matter sometimes illustrates different components contained within, or connected with, different other components. Such depicted architectures are merely exemplary, and in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality s achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably culpable”, to each other to achieve the desired functionality. Specific examples of operably culpable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In general terms used herein, and especially to the appended claims (e.g., bodies of the appended claims) are generally intended as “open” terms (e.g., the term “including” should be interpreted as “including but not limited to” the term “having” should be interpreted as “having at least,” the term “includes” should be interpreted as “includes but is not limited to,” etc.). If a specific number of an introduced claim recitation is intended, such an intent will be explicitly recited to the claim, and to the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” fruits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled to the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, to general such a construction is intended in the sense one having skill in the art would understand the convention (e.g., “a system having at least one of A, B, and C” would include but not be limited to systems that have A atone, B atone, C atone, A and B together, A and C together. B and C together, and/or A, B, and C together, etc.). It will be further understood by those within the art that virtually arty disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

For any and all purposes, such as to terms of providing a written description, all ranges disclosed herein also encompass any and all possible subranges and combinations of subranges thereof. Any listed range can be easily recognized as sufficiently describing and enabling the same range being broken down too at least equal halves, thirds, quarters, fifths, tenths, etc. As a non-limiting example, each range discussed herein can be readily broken down too a lower third, middle third and upper third, etc. All language such as “up to,” “at least,” “greater than,” “less than,” and the like include the number recited and refer to ranges which can be subsequently broken down into subranges as discussed above. Finally, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments are possible. The various aspects and embodiments disclosed herein are for purposes of illustration and are not intended to be limiting, with the true scope and spirt being indicated by the following claims.

What is claimed is:

1. A method to detect hydration in a subject, the method comprising:
coupling a shielded ring resonator structure to an exterior surface of the subject, wherein the shielded ring resonator structure includes:
a ring resonator;
a feed structure separated from the ring resonator by a first gap and a second gap and configured to couple signals into and out of the ring resonator; and
a metallic shielded structure conjured to cover the first and second gaps and a portion of the resonator;
applying a test signal to the feed structure to cause the shielded ring resonator structure to resonate with a first frequency characteristic;
using the metallic shield structure to reduce a capacitive coupling, while the test signal is applied, between the exterior surface of the subject and the first and second gaps;
determining a quality factor of the shielded ring resonator structure that resonates; and
determining a hydration level of the subject based on the determined quality factor.

2. The method of claim 1, wherein coupling the shielded ring resonator structure to the exterior surface of the subject comprises disposing an insulative layer between the metallic shield structure and the exterior surface of the subject.

3. The method of claim 2, wherein the insulative layer includes at least one medical tape and a low-allergen-content tape.

4. The method of claim 1, wherein coupling the shielded ring resonator structure to the exterior surface of the subject comprises affixing the shielded ring resonator structure to a skin of a human being.

5. A shielded ring resonator structure conjured to be coupled to an exterior surface of a subject to detect hydration of the subject, the shielded ring resonator structure comprising:
a ring resonator;
a first feed structure separated from the ring resonator by a first gap and configured to couple signals into the ring resonator;
a second feed structure separated from the ring resonator by a second gap and configured to couple signals oil of the ring resonator; and
a first conductive shield structure configured to cover a first portion of the ring resonator and at least one of the first and second gaps such that a capacitive coupling between the exterior surface of the subject and at least one of the first and second gaps is reduced.

6. The shielded ring resonator structure of claim 5, wherein the first conductive shield structure is configured to cover both of the first and second gaps.

7. The shielded ring resonator structure of claim 5, further comprising a second conductive shield structure, wherein the first conductive shield structure is configured to cover the first portion of the ring resonator and the first gap, and wherein the second conductive shield structure is configured to cover the second gap and a second portion of the ring resonator.

8. The shielded ring resonator structure of claim 5, wherein the first conductive shielding structure is coupled to a reference potential.

9. The shielded ring resonator structure of claim 5, wherein the first conductive shield structure includes aluminum or copper.

10. The shielded ring resonator structure of claim 5, further comprising an insulative layer disposed between the first conductive shield structure and at least one of the first gap and the second gap.

11. The shielded ring resonator structure of claim 5, further comprising an insulative layer disposed between the first conductive shield structure and the exterior surface of the subject.

12. The shielded ring resonator structure of claim 11, wherein the insulative layer includes at least one of a medical tape and a low-allergen-content tape.

13. The shielded ring resonator structure of claim 10, further comprising another insulative layer disposed between the first conductive shield structure and the exterior surface of the subject, wherein:
the first conductive shield structure has a generally rectangular shape and has a width to a range between about 3 mm and about 100 mm, a length to a range between about 3 mm and about 100 mm, and a thickness in a range between about 1 μm and about 2500 μm.

14. A system configured to detect hydration to a subject, the system comprising:
a shielded ring resonator structure conjured to be coupled to an exterior surface of the subject, wherein the shielded ring resonator structure comprises:
a ring resonator;
a first feed structure separated from the ring resonator by a first gap and conjured to couple a first signal into the ring resonator;
a second feed structure separated from the ring resonator by a second gap and conjured to couple a second signal out of the ring resonator; and
a first conductive shield structure conjured to cover a first portion of the ring resonator and the first gap such that a capacitive coupling between the exterior surface of the subject and the first gap is reduced;
a second conductive shielding structure conjured to cover a second portion of the ring resonator and the second gap such that a capacitive coupling between the exterior surface of the subject and the second gap is reduced; and
a test controller coupled to the shielded ring resonator structure and configured to:
apply the first signal to the shielded ring resonator structure to cause the ring resonator to resonate with a first frequency characteristic;
determine a quality factor of the resonating ring resonator based on the second signal; and
determine a hydration level of the subject based on the determined quality factor.

15. The system of claim 14, wherein the first conductive shield structure is coupled to a reference potential.

16. The system of claim 14, wherein the first conductive shield structure is coupled to the second conductive shield structure.

17. The system of claim 14, wherein the first conductive shield structure includes aluminum or copper.

18. The system of claim 14, further comprising an insulative layer disposed between the first conductive shield structure and at least one of the first gap and the second gap.

19. The system of claim 14, further comprising an insulative layer disposed between the first conductive shield structure and the exterior surface of the subject

20. The system of claim 19, wherein the insulative layer includes at least one of a medical tape and a low-allergen-content tape.

21. The system of chin 14, wherein the subject is a human being and the exterior surface is skin.

22. A method to manufacture a shielded ring resonator structure, the method comprising:
constructing a ring resonator on a substrate;
disposing a first feed structure on the substrate such that a first gap separates the first feed structure and the ring resonator, wherein the first feed structure is configured to couple a first signal into the ring resonator;
disposing a second feed structure on the substrate such that a second gap separates the second feed structure and the ring resonator, wherein the second feed structure is conjured to couple a second signal out of the ring resonator; and
placing a first conductive shield structure to cover a first portion of the ring resonator and at least one of the first and second gaps such that a capacitive coupling between an exterior surface of a subject to which the shielded ring resonator structure is attached and at least one of the first and second gaps is reduced.

23. The method of claim 22, further comprising placing an insulative layer between the first conductive shield structure and at least one of the first gap and the second gap.

24. The method of claim 22, further comprising placing an insulative layer between the first conductive shield structure and the exterior surface of the subject.
